(19) United States
(12) Patent Application Publication
(10) Pub. No.: US 2024/0226236 A9
(43) Pub. Date: Jul. 11, 2024

(54) NERVE GROWTH FACTOR FUSION PROTEIN, PREPARATION METHOD AND USE THEREOF

(71) Applicant: Staidson (Beijing) Biopharmaceuticals Co., Ltd., Beijing (CN)

(72) Inventors: Qingshuang Zhang, Beijing (CN); Lei Ma, Beijing (CN); Ming Liu, Beijing (CN)

(21) Appl. No.: 18/535,927

(22) Filed: Dec. 11, 2023

Related U.S. Application Data
(63) Continuation of application No. 16/085,984, filed on Sep. 17, 2018, now Pat. No. 11,883,464.

(30) Foreign Application Priority Data
Mar. 18, 2016 (CN) 201610159303.7

(51) Int. Cl. A61K 38/18; A61P 25/00; C07K 14/48; C12N 15/85

(52) U.S. Cl. CPC A61K 38/18; A61K 38/185; A61P 25/00; C07K 14/48; C12N 15/85; A61K 38/00

(57) ABSTRACT

The present disclosure relates to the field of biopharmaceuticals and provides a nerve growth factor (NGF) fusion protein and a preparation method and use thereof. The fusion protein has a general formula represented by A-B or A-L-B, wherein A is a nerve growth factor, L is a linker peptide, and B is an Fc moiety of IgG, or an analogue of the Fc moiety of IgG, or a fragment of the Fc moiety of IgG. The fusion protein of the present disclosure has the following advantages over a wild-type NGF: higher biological activity, a half-life extended more than 17 times, greatly reduced administration frequency, and significantly increased efficacy.

### CROSS REFERENCE TO RELATED APPLICATIONS

This application is a continuation application of U.S. patent application Ser. No. 16/085,984, filed on Sep. 17, 2018, which is a U.S. National Phase Application of PCT International Application Number PCT/CN2017/077025, filed on Mar. 17, 2017, designating the United States of America and published in the Chinese language, which is an International Application of and claims the benefit of priority to Chinese Patent Application No. 201610159303.7, filed on Mar. 18, 2016. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled “SeqList-DRGN005-001C1” created on Dec. 11, 2023, which is approximately 57,556 bytes in size. The information in the electronic format of the Sequence Listing is expressly incorporated herein by reference in its entirety.

### TECHNICAL FIELD

The disclosure relates to a nerve growth factor fusion protein, a preparation method and use thereof, and belongs to the field of biopharmaceutics.

### BACKGROUND

Nerve Growth Factor (NGF) is the first neurotrophic factor discovered in mouse sarcoma cells by Italian scientist Levi-Monticini in 1953. NGF is a neuronal growth regulator having a dual biological function of neuron nutrition and promoting neurite growth, which plays an important regulatory role in the development, differentiation, growth, regeneration, and expression of functional properties of central and peripheral neurons. NGF includes three subunits of α, β, and γ. The β subunit is an active region, which is formed by combining two single chains through a non-covalent bond. At present, a number of NGF products have been marketed at home and abroad, and are mainly used for the treatment of nervous system dysplasia, including amblyopia, neuroma, various nerve injuries and nervous system diseases.

As a protein drug, activity of NGF promoting nerve growth is mainly in a β-NGF having a sedimentation coefficient of 2.5S, a molecular weight of 13.5 Kd and being easily filtered by glomerulus during metabolism, resulting in a short half-life in vivo. Studies have shown that mice were intramuscularly administered with β-NGF drug, T½(β)=2.2 h, Tmax=0.5 h, and the frequency of injection was once a day. Due to the adverse reactions of a pain at the injection site or in the lower limb on the injection side during an NGF injection, the reduction in the number and frequency of administration to the patient is good for alleviating the adverse reactions of the patient. One of the solutions is to develop a long-acting and high biologically active NGF.

Protein drug modification is one of the research focuses on a long acting of current protein drugs, in which the construction of fusion proteins is an important strategy for protein modification. A gene of the target protein is linked end-to-end with a gene of a certain protein having a longer half-life and larger molecular weight, and the gene expression product (i.e., the fusion protein) is controlled by the same regulatory sequence, but it is still a clinically difficult problem to increase the biological activity of the protein drug while prolonging its half-life.

### SUMMARY

In order to solve the above problems, an object of the present application is to provide a nerve growth factor fusion protein. As compared with an original protein, the fusion protein not only has an enhanced biological activity, greatly prolonged half-life and reduced the number and frequency of administration to patients, thereby alleviating the patient's adverse reactions, but also has significantly improved the efficacy.

The present application provides a nerve growth factor fusion protein, represented by a general formula A-B or A-L-B, in which A is a nerve growth factor, L is a linker peptide, and B is an Fc moiety of IgG, an analogue of the Fc moiety of IgG, or a fragment of the Fc moiety of IgG.

IgG is an immunoglobulin having the highest content in serum. IgG is also an immunoglobulin having the longest half-life in serum among all immunoglobulins. Unlike other immunoglobulins, IgG may efficiently recycle after binding to an Fc receptor. There are four subclasses of IgG, i.e., G1, G2, G3, and G4, each having different effector functions. The Fc moiety of an immunoglobulin used herein has the common meaning of terms in the field of immunology. Specifically, the term “Fc moiety of an immunoglobulin” refers to an antibody fragment obtained by removing two antigen binding regions (Fab fragments) from an antibody. One method for removing Fab fragments is to digest the immunoglobulin with papain. Thus, the Fc moiety is formed by fragments having almost equal size of the constant regions of two heavy chains, in which the two heavy chains are associated by a non-covalent interactions and a disulfide bond. The Fc moiety may include a hinge region and extends through CH2 and CH3 domains to the C-terminal of the antibody.

According to the desired effect in vivo, the B moiety in the general formula of the nerve growth factor fusion protein may be an Fc moiety of any subclass of IgG or a mutant thereof. Thus, the nerve growth factor fusion protein of the present application may include an intact Fc moiety of an immunoglobulin, a fragment of the Fc moiety of the immunoglobulin, or an analog thereof, that is fused to a nerve growth factor.

Due to the introduction of a sequence of the Fc moiety, it may affect the activity of NGF, and may also mediate antibody-dependent cytotoxicity and complement-dependent cytotoxicity. Therefore, in order to obtain a fusion protein having a high biological activity and a long half-life, it is necessary to screen or mutate the Fc sequences of various subclasses.

B in the general formula of the nerve growth factor fusion protein is an Fc moiety of IgG1, an analog of the Fc moiety of IgG1, or a fragment of the Fc moiety of IgG1; preferably, the Fc moiety of IgG1 includes CH2 and CH3 regions, including a hinge region; and more preferably, the Fc moiety of IgG1 has an amino acid sequence of SEQ ID NO: 5.

Preferably, B in the nerve growth factor fusion protein is preferably an analog of the Fc moiety of IgG1. The analog of the Fc moiety includes an engineering modification, and the engineering modification may be a site mutation associated with antibody dependent cell-mediated cytotoxicity (ADCC)/complement dependent cytotoxicity (CDC) activity, or a deglycosylation mutation. More preferably, the analog of the Fc moiety has an amino acid sequence of SEQ ID NO: 7. Further preferably, the analog of the Fc moiety has an amino acid sequence of SEQ ID NO: 7 with the first 5 amino acids deleting at N-terminal.

The nerve growth factor is a wild type human nerve growth factor. Any human nerve growth factor may be a part of the nerve growth factor fusion protein of the present application, as long as the human nerve growth factor itself may bind to the human nerve growth factor receptor and induces a signal transmission through the human nerve growth factor receptor.

The nerve growth factor is an analog of a human nerve growth factor, and generally preferably a human nerve growth factor having no more than 6 amino acid mutation sites, even more preferably, a human nerve growth factor having no more than 5 amino acid mutation sites, and most preferably a human nerve growth factor having no more than 4, 3 or 2 amino acid mutation sites.

The human nerve growth factor is a derivative of a human nerve growth factor. The term “a derivative of a human nerve growth factor” herein refers to a molecule having an amino acid sequence of a human nerve growth factor or an analog of the human nerve growth factor, but also having an additional chemical modification at one or more of amino acid side groups, alpha carbon atoms, terminal amino groups or terminal carboxyl groups. The chemical modifications include, but are not limited to, adding chemical moieties, creating new bonds, and removing chemical moieties. The modification at amino acid side groups includes, but is not limited to, an acylation of an epsilon amino group of lysine, an N-alkylation of arginine, histidine or lysine, an alkylation of carboxyl of glutamic acid or aspartic acid, and a deamination of glutamine or asparagine. The modification at terminal amino groups includes, but is not limited to, deamination, N-lower alkyl, N-di-lower alkyl, and N-acyl modifications. The modification at terminal carboxyl groups includes, but is not limited to, amide, lower alkyl acyl, dialkyl amide, and lower alkyl ester modifications. The lower alkyl group is a C1-C4 alkyl group. In addition, one or more side groups or terminal groups may be protected by a protecting group known to a person skilled in the field of chemistry. An alpha carbon of an amino acid may be mono- or di-methylated.

Many active fragments, analogs and derivatives of the human nerve growth factor are well known in the art, and any one of these analogs and derivatives may be a part of the nerve growth factor fusion protein of the present application. Some examples of new analogs of the human nerve growth factor as well as analogs and derivatives of the human nerve growth factor well known in the art are provided herein.

The human nerve growth factor has preferably an amino acid sequence of any one of SEQ ID NO: 1, SEQ ID NO: 2, SEQ ID NO: 13 to SEQ ID NO: 30 in the sequence listing.

The human nerve growth factor of the present application includes a series of nerve growth factor mutants (i.e., recombinant hNGFs), which are capable of alleviating side effects such as pain and are even painless. These nerve growth factor mutants have an amino acid sequence of any one of SEQ ID NO: 2 and SEQ ID NO: 13 to SEQ ID NO: 30 in the sequence listing, respectively.
F12E: has an amino acid sequence of SEQ ID NO: 2 (i.e., the F12E substitution corresponds to F133E as set forth in SEQ ID NO: 2);
K32G: has an amino acid sequence of SEQ ID NO: 13;
K32L: has an amino acid sequence of SEQ ID NO: 14;
K32Y: has an amino acid sequence of SEQ ID NO: 15;
R59L: has an amino acid sequence of SEQ ID NO: 16;
R59A: has an amino acid sequence of SEQ ID NO: 17;
D65A: has an amino acid sequence of SEQ ID NO: 18;
D65G: has an amino acid sequence of SEQ ID NO: 19;
K74L: has an amino acid sequence of SEQ ID NO: 20;
K88F: has an amino acid sequence of SEQ ID NO: 21;
K88L: has an amino acid sequence of SEQ ID NO: 22;
K88E: has an amino acid sequence of SEQ ID NO: 23;
K88G: has an amino acid sequence of SEQ ID NO: 24;
Q96E: has an amino acid sequence of SEQ ID NO: 25;
R114V: has an amino acid sequence of SEQ ID NO: 26;
R114F: has an amino acid sequence of SEQ ID NO: 27;
R114G: has an amino acid sequence of SEQ ID NO: 28;
R114L: has an amino acid sequence of SEQ ID NO: 29; and
F101A: has an amino acid sequence of SEQ ID NO: 30.

L is a glycine-rich peptide or a peptide having a sequence [SEQ ID NO: 46]n, in which n is 1, 2, 3, 4, 5 or 6. Preferably, the function and stability in vivo of the nerve growth factor fusion protein of the present application is optimized by the addition of a linker peptide (L in the general formula) to prevent potential undesired domain interactions. Although these linker peptides may be of any length and consist of any combination of amino acids, and their lengths preferably do not exceed the length necessary to prevent undesired domain interactions and/or to optimize biological function and/or stability. The linker peptide is preferably enriched in serine-glycine, and preferably is not more than 30 amino acids in length. The linker peptide is more preferably not more than 20 amino acids in length, and more preferably not more than 15 amino acids in length. A preferred linker peptide includes a repetition of the sequence SEQ ID NO: 46. Preferably, there are 2 to 6 repetitions of this sequence. Even more preferably, there are 3 to 4 repetitions of this sequence. The most preferred sequence of the linker peptide is SEQ ID NO: 47. That is, the most preferred linker peptide L is [SEQ ID NO: 46]3.

Preferably, the nerve growth factor fusion protein has an amino acid sequence of SEQ ID NO: 10.

The present application also encompasses a polynucleotide sequence, encoding the above-mentioned nerve growth factor fusion protein.

An expression vector includes the nucleotide sequence.

The expression vector is a DNA vector or a viral vector.

The DNA vector is selected from the group consisting of a DNA plasmid vector, a liposome bound thereto, a molecular conjugate bound thereto, and a polymer bound thereto; and preferably, the DNA plasmid vector is a eukaryotic expression vector; and the viral vector is selected from the group consisting of an adeno-associated virus vector, a lentiviral vector and an adenoviral vector.

A method for preparing a nerve growth factor fusion protein includes: transforming the above-mentioned expression vector into a host cell, and culturing a resultant recombinant cell to express the expression vector, so as to obtain the nerve growth factor fusion protein.

A host cell includes the expression vector.

The host cell is a mammalian cell.

The mammalian cell is a Chinese hamster ovary cell, a human embryonic kidney 293 cell, a COS cell or a Hela cell.

Provided is a pharmaceutical composition, comprising a pharmaceutically acceptable excipient, and one or more of the above-mentioned nerve growth factor fusion protein, the above-mentioned expression vector, and the above-mentioned host cell.

The medicament of the present application may be prepared into various forms such as an injection, a capsule, a tablet or powder, and the medicament having the above various dosage forms may be prepared according to a conventional method in the field of pharmacy.

The pharmaceutical composition is preferably an injection comprising a pharmaceutically acceptable excipient and the above-mentioned nerve growth factor fusion protein.

Provided is use of the nerve growth factor fusion protein in the preparation of a medicament for treating a nervous system disease. The nervous system disease refers to a disease associated with neuronal degeneration or injury in the central and/or peripheral nervous system. Specific examples of the nervous system diseases include, but are not limited to, Alzheimer's disease, Parkinson's disease, Huntington's disease, stroke, ALS, peripheral neuropathy, and other disorders characterized by necrosis or loss of neuron, regardless central neuron, peripheral neuron, or motor neuron, except treating nerve damage caused by trauma, burns, kidney failure, or injury. For example, peripheral neuropathy associated with certain conditions is such as a neuropathy associated with diabetes, AIDS or chemotherapy.

The medicament for treating a nervous system disease prepared by a nerve growth factor fusion protein may be administered to a patient. The exact dosage will depend on the disease to be treated, and may be determined by one skilled in the art using known techniques. Additionally, as is known in the art, an adjustment needs to be made based on age, weight, general health, gender, diet, time of administration, drug interaction, and severity of the disease, and may be determined by one skilled in the art through routine experimentation. The patient mentioned herein includes humans, and other animals and organisms. Therefore, these methods may be used for treating human and livestock.

The administration of the medicament for treating a nervous system disease prepared by the nerve growth factor fusion protein of the present application may be carried out by various methods, including, but not limited to, oral, subcutaneous, intravenous, intracerebral, intranasal, transdermal, intraperitoneal, intramuscular, intrapulmonary, vaginal, rectal, and intraocular administrations. Under some circumstances, such as treating a wound, it may be applied directly in a form of a solution or spray.

The pharmaceutical composition of the present application includes the nerve growth factor fusion protein in a form suitable for administration to a patient. In a preferred example, the pharmaceutical composition is in a water soluble form, and may include, for example, a carrier, a excipient, a stabilizer, a buffer, a salt, an antioxidant, a hydrophilic polymer, an amino acids, a carbohydrate, an ionic or nonionic surfactant, polyethylene glycol, propylene glycol or the like. The medicament prepared by the nerve growth factor fusion protein may also be implanted in a sustained release form by techniques known in the art or embedded in a microcapsule form.

Provided is use of the nerve growth factor fusion protein in the preparation of a medicament for effectively reducing weight.

The present application has the following advantages: as compared with the wild type NGF, the nerve growth factor fusion protein of the present application has a higher biological activity, the half-life may be extended by 17 times or more, thus greatly reducing the administration frequency, and meanwhile the efficacy is significantly increased.

The present application will be further described hereinafter in conjunction with drawings and specific examples, which are not intended to limit the scope of the present application. All equivalent substitutions in the art in accordance with the present application fall into the scope of the present application.

### BRIEF DESCRIPTION OF THE DRAWINGS

FIG. 1 is a color developing map of Western Blot of a purified sample of rhNGF-Fc fusion protein in Example 2.

FIG. 2 is a detection result of the in vitro activity of the fusion protein in Example 3.

FIG. 3 is a detection result of in vitro activity of the fusion protein in Example 3.

FIG. 4 is a detection result of in vivo half-life of the fusion protein in Example 4.

### DETAILED DESCRIPTION

The examples described hereinafter in conjunction with the drawings are illustrative, and intended to illustrate the present application, but is not to be construed as limiting the present application. The techniques or conditions not specified in the examples are performed in accordance with techniques or conditions described in the literature in the art or in accordance with the product specifications. All reagents or instruments that do not specify the manufacturer are commercially available common products.

### Example 1: Preparation of Expression Plasmid of the Fusion Protein

(1) Construction of Expression Plasmid of rhNGF-Fc1 Fusion Protein

A nucleotide sequence SEQ ID NO: 3 encoding human β-NGF was synthesized, and amplified as a template by PCR using primers F1/R1 to obtain a NGF fragment including a Not I restriction enzyme site at the 5′ end. A nucleotide sequence SEQ ID NO: 8 encoding human IgG1-Fc was synthesized and amplified as a template by PCR using primers F2/R2 to obtain an Fc fragment including the Age I restriction enzyme site at the 3′ end. The PCR products of the NGF fragment and of the Fc fragment were mixed and amplified as templates by PCR using primers F1/R2 to obtain an NGF-Fc fusion gene fragment and recover the NGF-Fc fusion gene fragment. Then, the NGF-Fc fusion gene fragment and plasmid pcDNA3.1 (purchased from Invitrogen (Shanghai) Trading Co., Ltd.) were respectively subjected to double digestion with Age I and Not I, recovery, ligation and transformation to obtain an expression plasmid of pcDNA3.1-rhNGF-Fc1.

F1:
(SEQ ID No: 31)
ATTTGCGGCCGCGCCACCATGACCATGTTG.
R1:
(SEQ ID No: 32)
GAGTTTTGTCACAAGATTTGGGCTCGGCTCTTCTCACAGCCTTCCTGCTG.
F2:
(SEQ ID No: 33)
CAGCAGGAAGGCTGTGAGAAGAGCCGAGCCCAAATCTTGTGACAAAACTC.
R2:
(SEQ ID No: 34)
GATTGTCGATCATTACTAACCGGTTCATTTACCCGGGGACAGG.

(2) Construction of Expression Plasmid of rhNGF-Li-Fc1 Fusion Protein

A nucleotide sequence SEQ ID NO: 3 encoding human β-NGF was synthesized, and amplified as a template by PCR using primers F1-2/R1-2 to obtain a NGF fragment including a Not I restriction enzyme site at the 5′ end. A combined sequence of a nucleotide sequence SEQ ID NO: 35 encoding a linker (G₄S)₃ and a nucleotide sequence SEQ ID NO: 9 encoding human IgG1-Fc was synthesized and amplified as a template by PCR using primers F2-2/R2-2 to obtain an Fc fragment including the Age I restriction enzyme site at the 3′ end. The PCR products of the NGF fragment and of the Fc fragment were mixed and amplified as templates by PCR using primers F1-2/R2-2 to obtain an NGF-Fc fusion gene fragment and recover the NGF-Fc fusion gene fragment. Then, the NGF-Fc fusion gene fragment and a plasmid pcDNA3.1 (purchased from Invitrogen (Shanghai) Trading Co., Ltd.) were respectively subjected to double digestion with Age I and Not I, recovery, ligation and transformation to obtain a expression plasmid of pcDNA3.1-rhNGF-li-Fc1.

F1-2:
(SEQ ID No: 36)
GCCCTCTAGACTCGAGCGGCCGCGCCACCATGACCATGTTGT
TCTACACTC.
R1-2:
(SEQ ID No: 37)
CGCCGGAGCCGCCACCGCCGGCTCTTCTCACAGCCTTCCTG.
F2-2:
(SEQ ID No: 38)
CAGGAAGGCTGTGAGAAGAGCCGGCGGTGGCGGCTCCGGCG.
R2-2:
(SEQ ID No: 39)
GATTGTCGATCATTACTAACCGGTTCATTTACCCGGGGACAG
GGAGAGGC.

(3) Construction of Expression Plasmid of rhNGF(F12E)-Fc1 Fusion Protein

The expression plasmid of fusion protein pcDNA3.1-rhNGF-Fc1 was amplified as a template by PCR using mutant primers F12E-F/F12E-R, recovered, digested with a Dpn I restriction enzyme and then transformed, to obtain a expression plasmid of pcDNA3.1-rhNGF(F12E)-Fc1.

F12E-F:
(SEQ ID No: 40)
TTCCACAGGGGCGAAGAGTCGGTGTGTGACAGT.
F12E-R:
(SEQ ID No: 41)
ACTGTCACACACCGACTCTTCGCCCCTGTGGAA.

(4) Construction of Expression Plasmid of rhNGF-Fc4 Protein

First, the pcDNA3.1-rhNGF-Fc1 fusion gene was amplified as a template by PCR using primers 1F/1R to obtain a vector backbone containing the NGF sequence. A human IgG4-Fc nucleotide sequence SEQ ID NO: 12 was synthesized and amplified as a template by PCR using primers PAA-F/PAA-R to obtain an IgG4-Fc-containing fragment. Then, the two PCR products were seamlessly linked and then transformed.

1F:
(SEQ ID No: 42)
GTCTCTGGGTAAATAAACCGGTTAGTAATGATC
1R:
(SEQ ID No: 43)
GACCATATTTGGACTCGGCTCTTCTCACAGC.
PAA-F:
(SEQ ID No: 44)
CTGCCCAGCACCTGAGGCTGCGGGGGGACCATCAGTCTTC.
PAA-R:
(SEQ ID No: 45)
GAAGACTGATGGTCCCCCCGCAGCCTCAGGTGCTGGGCAG.

Experimental Results:

Positive clones were picked up for sequencing, and the result confirmed that the gene sequences expressed by the rhNGF-Fc1, rhNGF-li-Fc1, rhNGF(F12E)-Fc1 and rhNGF-Fc4 fusion proteins were correct.

### Example 2: Expression and Purification of Fusion Protein

1. CHO Cell Expressing the Fusion Protein

The cultured CHO cells were suspended, and were adjusted to a cell density of 2.5×10⁶/ml after washing once with DMEM medium. Each 300 ml of cells for transfection required the following steps: 300 μg of expression plasmid was diluted with 10 ml of Opti-RPO (Invitrogen), 1.5 ml of PEI is diluted with 6 ml of Opti-PRO (1 mg/ml), after dilution they were thoroughly mixed respectively and left for 5 min. Then PEI dilution was added to the plasmid dilution, mixed and incubated for 10 min at room temperature. The PEI-plasmid mixture was then slowly added to 150 ml of resuspended cells, and incubated under shaking in a 6% carbon dioxide incubator at 37° C. and 105 rpm for 4 h. An equal volume of 150 ml of expression medium EX-CELL Advanced CHO Fed-batch medium (sigma) and 1 mM sodium valproate (0.5 mol/L) were added, and incubated under shaking in a 6% carbon dioxide incubator at 32° C. and 120 rpm for 18-24 h. 30 ml of supplement Sheff-CHO Plus ACF (Kerry Sheffield) (50 g/L) was added, and the supernatant was harvested after 4 days.

2. Purification of Fusion Protein by Protein a Affinity Column

The supernatant was collected from the cell culture, and the cells and fragments were removed by filtration through a 0.45 μm filter. The supernatant was loaded to a prepared Protein A column, purified with 20 mM PB+0.15 M NaCl solution (pH 7.2), further eluted with 50 mM citrate buffer (pH 3.4), and the protein sample was collected and immediately neutralized to a pH of 6.8 by adding 2 mol/L Tris solution.

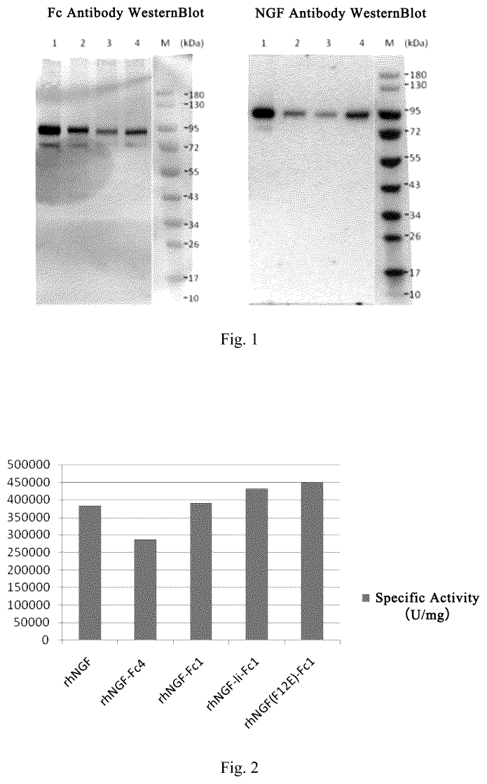

The purified samples were subjected to Western Blot detections of NGF antibody and Fc antibody. The results are shown in FIG. 1, in which samples corresponding to lanes 1 to 4 are rhNGF-Fc1, rhNGF-li-Fc1, rhNGF(F12E)-Fc1 and rhNGF-Fc4 fusion proteins, respectively.

### Example 3: Detection of In Vitro Activity of Fusion Protein

The detailed operation method was performed in accordance with the method in Example 1 of a patent entitled “Method for Quantitatively Measuring Nerve Growth Factor Activity” with a publication number of CN103376248A, and the specific activity and molar specific activity results are shown in the following table.

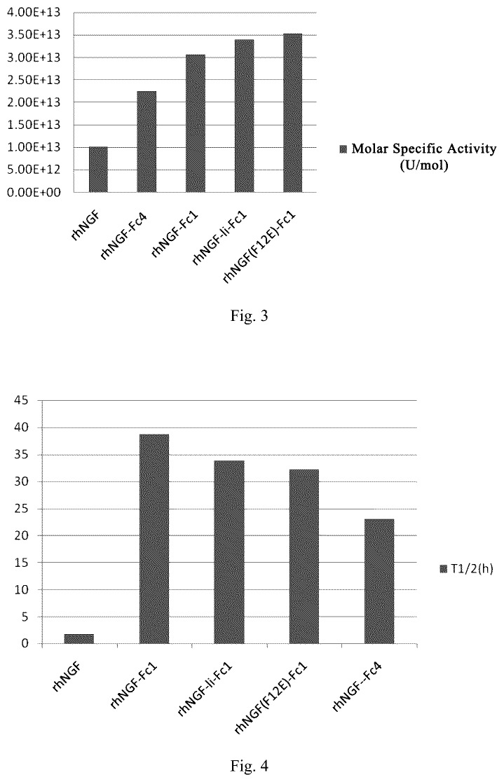

The experimental results were shown in Table 1, FIG. 2 and FIG. 3.

The specific activity of rhNGF-Fc1, rhNGF-li-Fc1, rhNGF(F12E)-Fc1 fusion proteins was basically the same as that of rhNGF, while the specific activity of rhNGF-Fc4 fusion protein was decreased.

According to the calculation of the molar specific activity converted by molecular weight, the molar specific activity of the NGF fusion protein of the IgG1-Fc subtype was higher than that of the corresponding rhNGF, and was also better than the molar specific activity of the rhNGF-Fc4 fusion protein.

TABLE 1

| | Test Sample | | | | |
|---|---|---|---|---|---|
| Activity | rhNGF | rhNGF-Fc4 | rhNGF-Fc1 | rhNGF-li-Fc1 | rhNGF(F12E)-Fc1 |
| Specific Activity | 383001.55 | 286921.8 | 390443.1 | 431625 | 448338.8 |
| (U/mg) | | | | | |
| Molar Specific | 1.03E+13 | 2.26E+13 | 3.07E+13 | 3.40E+13 | 3.53E+13 |
| Activity (U/mol) | | | | | |

### Example 4: Detection of the In Vivo Half-Life of Fusion Protein

1. Administration and Blood Collection

SD male rats weighted about 250 g were randomly divided into groups (4 rats per group), and the test samples were administered intramuscularly in a dose of 1 ml/kg according to the body weight of the rats. The test samples were rhNGF, rhNGF-Fc1, rhNGF-li-Fc1, rhNGF (F12E)-Fc1 and rhNGF-Fc4, respectively, and the dose was 1.2 nmol/kg. 0.15 ml of blood was collected from posterior orbital vein at different time points before and after administration. The collected blood was immediately placed in a 1.5 ml EP tube pre-loaded with 20 ul of 1% heparin sodium, and the mixture was immediately inverted and mixed several times, followed by centrifuge at 4000 rpm and 4° C. for 20 min. The supernatant plasma was collected and frozen at −80° C.

2. Elisa Detection

The concentration of the drug in the plasma was detected by using hNGF elisa kit (purchased from Beijing sinobiological Co., Ltd., item number: SEK11505).

3. Results

According to the results of the concentration of hNGF in the plasma by Elisa detection, the half-life of the test sample was fitting calculated by the non-compartmental model method (NCA) with WinNonlin 6.2 software. The obtained T½ of the rhNGF, rhNGF-Fc1, rhNGF-li-Fc1, rhNGF(F12E)-Fc1 and rhNGF-Fc4 proteins were 1.8 h, 38.75 h, 33.83 h, 32.24 h and 23.1 h, respectively. As compared with the in vivo half-life of rhNGF of 1.8 h, the in vivo half-life of the fusion proteins rhNGF-Fc1, rhNGF-li-Fc1 and rhNGF(F12E)-Fc1 was significantly prolonged, thereby achieving 32 hours or more and prolonging by more than 17 times. As compared with the in vivo half-life of rhNGF-Fc4, the in vivo half-life of the fusion proteins rhNGF-Fc1, rhNGF-li-Fc1 and rhNGF(F12E)-Fc1 were greatly prolonged, thereby reducing the metabolic rate of rhNGF. The results are shown in Table 2 and FIG. 4.

TABLE 2

| Test Sample | T_{1/2}(h) |
|---|---|
| rhNGF | 1.8 |
| rhNGF-Fc1 | 38.75 |
| rhNGF-li-Fc1 | 33.83 |
| rhNGF(F12E)-Fc1 | 32.24 |
| rhNGF--Fc4 | 23.1 |

### Example 5: Detection of Efficacy of Fusion Protein

Male Kunming mice were selected, and the pain threshold (basal pain threshold, taking the licking rear feet as an observational indication) was determined by hot plate method (54-55° C.), and male Kunming mice with a pain threshold of more than 30 s were excluded, to obtain eligible mice. Mouse was anesthetized with ether, and then a mouse model of sciatic nerve injury using a nerve clamping method was established, while the sham operation group was only separated the sciatic nerve, but without a clamp.

Mice were divided into three groups: a sham operation group, an injury control group (normal saline) and an experimental group (rhNGF, rhNGF-Fc1, rhNGF-li-Fc1, rhNGF(F12E)-Fc1 and rhNGF-Fc4 fusion proteins), in which each group includes 5 mice.

During the operation, 50 μl of protein sample having the corresponding concentration or normal saline (control) was topically added dropwise, the skin was sutured; 0.5E+11 AU/mol of protein sample or normal saline was intraperitoneally injected to the mice, and the pain threshold of each mouse (the latency of the licking rear feet of the mouse, i.e., the pain threshold (s)) was determined before the surgery and on day 1, day 3, day 5 and day 10 after the surgery, respectively.

According to the pain threshold, the increase in pain threshold of the mouse on day 10 was calculated according to the following formula:

Increase in pain threshold (%)=Pain threshold on day 10 after injury−Pain threshold before injury/Pain threshold before injury×100%

The experimental results are shown in Table 3 below. As can be seen from the pain threshold value, the pain threshold of the sciatic nerve injured mouse increased on day 1 to day 3 significantly, and the pain threshold gradually recovered over time. The increase in pain threshold of the NGF fusion protein sample of IgG1-Fc and IgG4-Fc subtypes on day 10 after a single-dose administration was significantly better than those of the rhNGF group and the injury control group, and the recovery effect of the NGF fusion protein of IgG1-Fc subtype was significantly better than that of the fusion protein of IgG4-Fc subtype.

TABLE 3
Effect of Fusion Protein on Pain Threshold
Changes in Mouse After Sciatic Nerve Injury

| | | | | | | Increase in |
|---|---|---|---|---|---|---|
| | | | | | | Pain |
| Experimental | Before | | | | | Threshold |
| Treatments | Injury | After Injury (s) | | | | on Day 10 |
| (AU/mol) | (s) | 1 d | 3 d | 5 d | 10 d | (%) |
| Sham Operation | 15.2 | 18.6 | 17.5 | 18.4 | 18.7 | 0 |
| Group | | | | | | |
| Injury Control | 17.3 | 56.8 | 57.4 | 51.9 | 36.5 | 111.0 |
| Group | | | | | | |
| rhNGF | 17.1 | 48.8 | 54.6 | 47.5 | 33.9 | 98.2 |
| rhNGF-Fc1 | 17.0 | 59.7 | 56.4 | 44.2 | 27.1 | 59.4 |
| rhNGF-li-Fc1 | 16.9 | 55.7 | 57.1 | 48.6 | 29.4 | 60.4 |
| rhNGF(F12E)-Fc1 | 15.8 | 54.2 | 51.7 | 44.5 | 25.3 | 60.1 |
| rhNGF-Fc4 | 18.6 | 60.1 | 53.9 | 50.1 | 32.1 | 72.6 |

1. (canceled)

2. A nucleic acid, which comprises a nucleotide sequence encoding a nerve growth factor fusion protein, wherein the nerve growth factor fusion protein comprises a general formula A-B or A-L-B, wherein:
A is a human nerve growth factor,
L is a linker peptide, and
B is an Fc moiety of IgG, a mutant of the Fc moiety of IgG, or a fragment of the Fc moiety of IgG,
wherein the human nerve growth factor comprises F12E with reference to the amino acid position set forth in a wild-type human nerve growth factor.

3. The nucleic acid according to claim 2, wherein B is an Fc moiety of IgG1, a mutant of the Fc moiety of IgG1, or a fragment of the Fc moiety of IgG1, wherein the mutant of the Fc moiety comprises a site mutation associated with antibody dependent cell-mediated cytotoxicity (ADCC)/complement dependent cytotoxicity (CDC) activity, or a deglycosylation mutation.

4. The nucleic acid according to claim 3, wherein the mutant of Fc moiety of IgG1 has an amino acid sequence of SEQ ID NO: 7, or an amino acid sequence of SEQ ID NO: 7 with the first 5 amino acids deleting at N-terminal.

5. The nucleic acid according to claim 2, wherein the wild-type human nerve growth factor comprises an amino acid sequence of SEQ ID NO: 1.

6. The nucleic acid according to claim 2, wherein the human nerve growth factor comprises an amino acid sequence of SEQ ID NO: 2.

7. The nucleic acid according to claim 2, wherein L is a glycine-rich peptide or a peptide having a sequence [SEQ ID NO: 46]n, wherein n is 1, 2, 3, 4, 5 or 6.

8. The nucleic acid according to claim 2, wherein L is not more than 30 amino acids in length.

9. The nucleic acid according to claim 2, wherein the nerve growth factor fusion protein comprises an amino acid sequence of SEQ ID NO: 10.

10. The nucleic acid according to claim 1, wherein the nucleic acid comprises a nucleotide sequence of SEQ ID NO: 12.

11. An expression vector, comprising the nucleic acid of claim 2.

12. The expression vector according to claim 11, wherein the expression vector is selected from the group consisting of a DNA vector and a virus vector.

13. The expression vector according to claim 12, wherein the DNA vector is selected from the group consisting of a DNA plasmid vector, a liposome bound thereto, a molecular conjugate bound thereto, and a polymer bound thereto.

14. A method for preparing a nerve growth factor fusion protein, comprising transfecting the expression vector of claim 11 into a host cell, and culturing the resulting recombinant cell to express the expression vector.

15. A host cell, comprising the nucleic acid of claim 2 or an expression vector comprising the nucleic acid of claim 2.

16. The host cell according to claim 15, wherein the host cell is a mammalian cell.

17. A pharmaceutical composition, comprising: the nucleic acid of claim 2 or an expression vector comprising the nucleic acid, or a host cell comprising the expression vector, and a pharmaceutically acceptable excipient.

18. A method of using a nerve growth factor fusion protein to treat or inhibit a nervous system disease or to reduce weight, comprising administering the nerve growth factor fusion protein to a subject in need thereof,
wherein the nerve growth factor fusion protein comprises a general formula A-B or A-L-B,
wherein: A is a human nerve growth factor,
L is a linker peptide, and
B is an Fc moiety of IgG, a mutant of the Fc moiety of IgG, or a fragment of the Fc moiety of IgG,
wherein the human nerve growth factor comprises F12E with reference to the amino acid position set forth in a wild-type human nerve growth factor.

19. The method according to claim 18, wherein B is an Fc moiety of IgG1, a mutant of the Fc moiety of IgG1, or a fragment of the Fc moiety of IgG1, wherein the mutant of the Fc moiety comprises a site mutation associated with antibody dependent cell-mediated cytotoxicity (ADCC)/complement dependent cytotoxicity (CDC) activity, or a deglycosylation mutation.

20. A method of using the nucleic acid of claim 2, an expression vector comprising the nucleic acid, or a host cell comprising the expression vector to treat or inhibit a nervous system disease or to reduce weight in a subject, comprising:
administering the nucleic acid of claim 2, the expression vector comprising the nucleic acid or the host cell comprising the expression vector to a subject in need thereof.


SEQUENCE LISTING (47 sequences)

SEQ ID NO: 1 - protein, 241 aa, Homo sapiens
MTMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - protein, 241 aa, Homo sapiens
MTMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 3 - DNA, 723 bp, Homo sapiens
  source 1..723: mol_type = other DNA
atgaccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcc

SEQ ID NO: 4 - DNA, 723 bp, Homo sapiens
  source 1..723: mol_type = other DNA
atgaccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcc

SEQ ID NO: 5 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 699 bp, Homo sapiens
  source 1..699: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 7 - protein, 231 aa, Homo sapiens
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 696 bp, Homo sapiens
  source 1..696: mol_type = other DNA
gagcccaaatcttgtgacaaaactcacacatgcccaccgtgcccagcacctccagtcgcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 9 - DNA, 681 bp, Homo sapiens
  source 1..681: mol_type = other DNA
gacaaaactcacacatgcccaccgtgcccagcacctccagtcgcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 10 - protein, 482 aa, Homo sapiens
MTMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRAGGGGSGGGGSGGGGSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1449 bp, Homo sapiens
  source 1..1449: mol_type = other DNA
atgaccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccggcggtggcggctccggcggtggcggctccggcggtggcggctccgacaaaactcacacatgcccaccgtgcccagcacctccagtcgcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 12 - DNA, 690 bp, Homo sapiens
  source 1..690: mol_type = other DNA
gagtccaaatatggtcccccatgcccaccctgcccagcacctgaggctgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 13 - protein, 120 aa, Homo sapiens
SSSHPIFHRGEFSVCDSVSVWVGDKTTATDIGGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 14 - protein, 120 aa, Homo sapiens
SSSHPIFHRGEFSVCDSVSVWVGDKTTATDILGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 15 - protein, 120 aa, Homo sapiens
SSSHPIFHRGEFSVCDSVSVWVGDKTTATDIYGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 16 - protein, 120 aa, Homo sapiens
SSSHPIFHRGEFSVCDSVSVWVGDKTTATDIKGKEVMVLGEVNINNSVFKQYFFETKCLDPNPVDSGCRGIDSKHWNSYCTTTHTFVKALTMDGKQAAWRFIRIDTACVCVLSRKAVRRA

SEQ ID NO: 17 - protein, 120 aa, Homo sapiens
SSSHPIFHRGEFSVCDSVSVWVGDKTTATDIKGKEVMVLGEVNINNSVFKQYFFETKCADPNPVDSGCRGIDSKHWNSYCTTTHTFVKALTMDGKQAAWRFIRIDTACVCVLSRKAVRRA

SEQ ID NO: 18 - protein, 120 aa, Homo sapiens
SSSHPIFHRGEFSVCDSVSVWVGDKTTATDIKGKEVMVLGEVNINNSVFKQYFFETKCRDPNPVASGCRGIDSKHWNSYCTTTHTFVKALTMDGKQAAWRFIRIDTACVCVLSRKAVRRA

SEQ ID NO: 19 - protein, 120 aa, Homo sapiens
SSSHPIFHRGEFSVCDSVSVWVGDKTTATDIKGKEVMVLGEVNINNSVFKQYFFETKCRDPNPVGSGCRGIDSKHWNSYCTTTHTFVKALTMDGKQAAWRFIRIDTACVCVLSRKAVRRA

SEQ ID NO: 20 - protein, 120 aa, Homo sapiens
SSSHPIFHRGEFSVCDSVSVWVGDKTTATDIKGKEVMVLGEVNINNSVFKQYFFETKCRDPNPVDSGCRGIDSLHWNSYCTTTHTFVKALTMDGKQAAWRFIRIDTACVCVLSRKAVRRA

SEQ ID NO: 21 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVFALTMDGKQAAWRFIRIDTACVCVLSRKAVRRA

SEQ ID NO: 22 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVLALTMDGKQAAWRFIRIDTACVCVLSRKAVRRA

SEQ ID NO: 23 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVEALTMDGKQAAWRFIRIDTACVCVLSRKAVRRA

SEQ ID NO: 24 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVGALTMDGKQAAWRFIRIDTACVCVLSRKAVRRA

SEQ ID NO: 25 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKEAAWRFIRIDTACVCVLSRKAVRRA

SEQ ID NO: 26 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSVKAVRRA

SEQ ID NO: 27 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSFKAVRRA

SEQ ID NO: 28 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSGKAVRRA

SEQ ID NO: 29 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSLKAVRRA

SEQ ID NO: 30 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRAIRIDTACVCVLSRKAVRRA

SEQ ID NO: 31 - DNA, 30 bp, synthetic construct
  misc_feature 1..30: note = Primer F1
  source 1..30: mol_type = other DNA
atttgcggccgcgccaccatgaccatgttg

SEQ ID NO: 32 - DNA, 50 bp, synthetic construct
  misc_feature 1..50: note = Primer R2
  source 1..50: mol_type = other DNA
gagttttgtcacaagatttgggctcggctcttctcacagccttcctgctg

SEQ ID NO: 33 - DNA, 50 bp, synthetic construct
  misc_feature 1..50: note = Primer F2
  source 1..50: mol_type = other DNA
cagcaggaaggctgtgagaagagccgagcccaaatcttgtgacaaaactc

SEQ ID NO: 34 - DNA, 43 bp, synthetic construct
  misc_feature 1..43: note = Primer R2
  source 1..43: mol_type = other DNA
gattgtcgatcattactaaccggttcatttacccggggacagg

SEQ ID NO: 35 - DNA, 45 bp, synthetic construct
  misc_feature 1..45: note = Linker
  source 1..45: mol_type = other DNA
ggcggtggcggctccggcggtggcggctccggcggtggcggctcc

SEQ ID NO: 36 - DNA, 51 bp, synthetic construct
  misc_feature 1..51: note = Primer F1-2
  source 1..51: mol_type = other DNA
gccctctagactcgagcggccgcgccaccatgaccatgttgttctacactc

SEQ ID NO: 37 - DNA, 41 bp, synthetic construct
  misc_feature 1..41: note = Primer R1-2
  source 1..41: mol_type = other DNA
cgccggagccgccaccgccggctcttctcacagccttcctg

SEQ ID NO: 38 - DNA, 41 bp, synthetic construct
  misc_feature 1..41: note = Primer F2-2
  source 1..41: mol_type = other DNA
caggaaggctgtgagaagagccggcggtggcggctccggcg

SEQ ID NO: 39 - DNA, 50 bp, synthetic construct
  misc_feature 1..50: note = Primer R2-2
  source 1..50: mol_type = other DNA
gattgtcgatcattactaaccggttcatttacccggggacagggagaggc

SEQ ID NO: 40 - DNA, 33 bp, synthetic construct
  misc_feature 1..33: note = Primer F12E-F
  source 1..33: mol_type = other DNA
ttccacaggggcgaagagtcggtgtgtgacagt

SEQ ID NO: 41 - DNA, 33 bp, synthetic construct
  misc_feature 1..33: note = Primer F12E-R
  source 1..33: mol_type = other DNA
actgtcacacaccgactcttcgcccctgtggaa

SEQ ID NO: 42 - DNA, 33 bp, synthetic construct
  misc_feature 1..33: note = Primer 1F
  source 1..33: mol_type = other DNA
gtctctgggtaaataaaccggttagtaatgatc

SEQ ID NO: 43 - DNA, 31 bp, synthetic construct
  misc_feature 1..31: note = Primer 1R
  source 1..31: mol_type = other DNA
gaccatatttggactcggctcttctcacagc

SEQ ID NO: 44 - DNA, 40 bp, synthetic construct
  misc_feature 1..40: note = Primer PAA-F
  source 1..40: mol_type = other DNA
ctgcccagcacctgaggctgcggggggaccatcagtcttc

SEQ ID NO: 45 - DNA, 40 bp, synthetic construct
  misc_feature 1..40: note = Primer PAA-R
  source 1..40: mol_type = other DNA
gaagactgatggtccccccgcagcctcaggtgctgggcag

SEQ ID NO: 46 - protein, 5 aa, synthetic construct
  REGION 1..5: note = Linker peptide
GGGGS

SEQ ID NO: 47 - protein, 15 aa, synthetic construct
  REGION 1..15: note = Linker peptide
GGGGSGGGGSGGGGS